(19) United States
(12) Patent Application Publication
(10) Pub. No.: US 2023/0355479 A9
(43) Pub. Date: Nov. 9, 2023

(54) Instrumented Medicine Dispenser

(71) Applicants: Charlie Selbin, New York, NY (US); Jakob Siden, New York, NY (US)

(72) Inventors: Charlie Selbin, New York, NY (US); Jakob Siden, New York, NY (US)

(21) Appl. No.: 17/398,859

(22) Filed: Aug. 10, 2021

Related U.S. Application Data
(60) Provisional application No. 63/083,061, filed on Sep. 24, 2020.

(51) Int. Cl. A61J 7/04; G16H 20/13

(52) U.S. Cl. CPC A61J 7/0481; G16H 20/13

(57) ABSTRACT

The present invention is directed to an instrumented medication dispenser with real-time vital sign monitoring in order to assist patients in managing their medicine intake. It provides various benefits such as assisting patients in maintaining the correct dosing of their medicines, but most important is the ability to monitor the patient's vital signs and provide real time updates to a doctor. It therefore assists patients in the event that a health issue arises, especially in instances where the health issue is related to medicine intake, and providing information and support to health care providers.

### RELATED APPLICATIONS

This application claims priority to U.S. Provisional Patent Application No. 63/083,061, filed on Sep. 24, 2020; the entirety of which is incorporated herein by reference.

### BACKGROUND OF THE INVENTION

A common problem for medical patients and caregivers is monitoring the usage of medications and enforcing compliance for medications regimen and schedules. Timely consumption of the required dosage and types of medication are of critical importance, as evidenced by the fact that, according to the World Health Organization, failing to take medications at the proper time in the proper dosage leads to 125,000 deaths per year. Some of the most critical and seemingly simple questions relating to compliance with medication regimens are: (1) “Did you take your medications today?” (2) “Did you take your medicine at the prescribed time?” and (3) “Did you take your medicine in the correct amount and/or the prescribed time?” The statistical responses to these questions are dependent on age, language barriers, socio-economic status, and conditions such as dementia and depression.

Overdosing of patients who rely on prescription drugs is a significant issue in public health. Such overdoses can occur either intentionally or unintentionally. In many cases, a patient either forgets that they have taken the drug earlier that day or makes an unfortunate decision in combining drugs.

Although various methods, including medication reminder software applications (apps), have been put forward to address these issues, the problem remains unresolved. In this respect, there are scores of medication reminder software apps and related services available, however, these often become simply glorified alarm clocks and quickly go from utility to annoying. With random snoozing and cancellations by patients, these solutions are full of false positives and lack adequate safety measures.

In contrast, what is needed is an automated and efficient method and system for monitoring the actual usage of medications, along with affording the ability to enforce compliance with medication regimens and schedules. More importantly, what is needed is a method and system that is additionally able to monitor vitals as well as allow the doctor to remotely monitor and change dosage.

### SUMMARY OF THE INVENTION

Accordingly, the present system provides an instrumented medication dispenser with real-time vital sign monitoring in order to assist patients in managing their medicine intake. It provides various benefits such as assisting patients in maintaining the correct dosing of their medicines, but most important is the ability to monitor the patient's vital signs and provide real time updates to a doctor. It therefore assists patients in the event that a health issue arises, especially in instances where the health issue is related to medicine intake, and providing information and support to health care providers.

As such one aspect of the present invention provides an instrumented medication dispenser for monitoring usage of medication and adaptively enforcing compliance with a medication schedule comprising: a user interface configured to receive a request for medication and to transmit that request to a processor; a medication locker configured to provide access to a dosage of medicine, the dosage of medicine having associated prescription information; and a computing device configured to receive the request for medication and to store information regarding the request, the computing device further configured to communicate a release request with the medication locker to release the dosage of medicine, wherein the computing device determines whether to transmit the release request based on a time of the request, the prescription information, and information regarding a previous request for the dosage.

### BRIEF DESCRIPTION OF THE FIGURES

Advantages of the present methods and related devices will be apparent from the following detailed description, which description should be considered in combination with the accompanying figures, which are not intended to limit the scope of the invention in any way.

Reference characters used in the drawings are referred to in the text, so the reader can look at the drawings and read the text to understand what the writer is discussing, much as would be the case with a well written instruction manual on how to put something together requiring assembly.

FIG. 1 is a schematic illustration of a system in accord with the present disclosure.

FIG. 2 illustrates a flowchart that illustrates a user interacting with a device in accord with the present disclosure.

FIG. 3 illustrates a flowchart that corresponds to a medicine being loaded into the dispenser.

FIG. 4 illustrates a flowchart of the system wherein there is additional interaction with a healthcare provider.

FIG. 5 illustrates a flowchart related to taking of patient's vital signs.

FIG. 6 illustrates an emergency response flowchart that corresponds to certain embodiments of the disclosed system.

### DETAILED DESCRIPTION OF THE INVENTION

The present disclosure relates generally to the longtime issue of drug overdose from pharmacy prescribed pills/medicines. In fact, the problem lies in the fact that once a drug is prescribed to someone and given to them, for example, in one of the standard orange pill bottles, there is nothing except a child lock to prevent a user from taking more pills than what they were told.

The system disclosed herein provides various benefits to patients and healthcare providers. In particular, the present system provides an instrumented medication dispenser with real-time vital sign monitoring in order to assist patients in managing their medicine intake. It provides various benefits such as assisting patients in maintaining the correct dosing of their medicines, but most important is the ability to monitor the patient's vital signs and provide real time updates to a doctor. It therefore assists patients in the event that a health issue arises, especially in instances where the health issue is related to medicine intake, and providing information and support to health care providers.

The combination of the security of the locked medication dispenser that does not allow patients to access their medications, as well as the patient's vital signs being monitored at a doctor's discretion is a novel and unique way of preventing overdose. Even more so, the real-time ability for doctors to safely and securely alter doses based on real-time vital sign monitoring opens the door to modern uses of prescription drugs. No longer will patients and doctors alike have to wait until the end of the prescription to understand the effects of the drugs. Rather, up-to-date information regarding the patient's health will be readily available consistently over the course of the prescription and intake of drugs.

The unique combination of vital-taking, the software used to communicate remotely with the user and doctor (and possibly 911), the ability for the doctor to remotely adjust medication consumption, the locked medication box, and the medication dispenser at each prescribed time is what differentiates this from anything else on the market.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

The present invention, including devices and methods will be described with reference to the following definitions that, for convenience, are set forth below. Unless otherwise specified, the below terms used herein are defined as follows:

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein to mean both “and” in the conjunctive form and “or” in the disjunctive form. In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense.

The language “based on” may be understood as not necessarily intended to convey an exclusive set of factors and may, instead, allow for existence of additional factors not necessarily expressly described, again, depending at least in part on context.

The language “one or more” as used herein, depending at least in part upon context, may be used to describe any feature, structure, or characteristic in a singular sense or may be used to describe combinations of features, structures or characteristics in a plural sense.

The term “instrumented” is used herein to describe equipping (something) with measuring instruments.

The language “medical professional” is used herein to describe any person or people licensed by a state or federal/national entity to prescribe medications. In certain embodiments, the medical professional is a doctor.

The term “medicine” and “medication, may be used interchangeably, and are art-recognized. In certain embodiments, the word “medication” may refer to pills, a powder, a liquid, a dosage of energy (such as electromagnetic radiation or audio emission), or other means of delivering health positive substance to an individual.

The term “user” as used herein, describes the user of the system, i.e., the person who will be receiving the medicine from the medicine dispensing system.

### II. Medication Dispenser

The present system provides an instrumented medication dispenser to assist patients in managing their medicine intake. It provides various benefits including: assisting patients in maintaining the correct dosing of their medicines, assisting patients in the event that a health issue arises, especially in instances where the health issue is related to medicine intake, and providing information and support to health care providers.

As such, one embodiment of the invention provides an instrumented medication dispenser for monitoring usage of medication and adaptively enforcing compliance with a medication schedule comprising:
a user interface configured to receive a request for medication and to transmit that request to a processor;
a medication locker configured to provide access to a dosage of medicine, the dosage of medicine having associated prescription information; and
a computing device configured to receive the request for medication and to store information regarding the request, the computing device further configured to communicate a release request with the medication locker to release the dosage of medicine, wherein the computing device determines whether to transmit the release request based on a time of the request, the prescription information, and information regarding a previous request for the dosage.

The invention illustrates a system based on the present disclosure. The system includes a lock mechanism, a medication dispenser, a series of sensors and tools that allows for a patient's vitals to be taken, a technology software that shares the patient's information with a doctor or medical professional, an alert system to reminds and ensures that the user is taking the necessary medications on-time, a user-friendly interface/screen, and a medicine loading system.

In certain embodiments of the present invention, the Medication Dispenser System e.g., a Pill Dispensing System, comprises a locking mechanism system. The lock mechanism may be controlled by, for example a physical lock and key system, an electronic lock, a username/password combination, or similar access control device. The lock mechanism may control various aspects of the system. The lock mechanism controls access to the medicine by the patient, the lock mechanism also controls access to the medication loading system. The loading system is used as an access point to deposit medicine into the medication locker of the system. In certain embodiments of the present invention there may be multiple medication lockers, e.g., pill lockers, within the system. In particular embodiments, the loading system allows a user of the system to deposit metadata associated with the medicine in electronic form into the device. This metadata may include, for example, information regarding the medicine formulation, the dosage associated with the medicine, contraindications associated with the medicine, and other information necessary to effectively run the system.

The systems of the present invention comprise a user interface that allows a user to enter and receive data to or from the system. The system further comprises a computing device that is integrated into the system to store data and to provide processing decisions. The computing device is connected to a networked communication system that transmits and receives messages from outside of the system. In particular embodiments, the networked communication system may, for example, use ethernet, 802.11, cellular communications or other means for external communications. The system further comprises a vital sign monitoring apparatus. In particular embodiments, this system may access and record vitals such as Heart Rate, Blood Pressure, Blood Sugar, Body Temperature, or similar vital signs as necessary.

In certain embodiments, the system may be used to monitor a patient's vitals in order to allow doctors and health care professionals to safely alter the strength or dosage of a patient's medication, e.g., completely remotely. The medications are immediately locked inside a medication dispensing unit, so patients will have zero access to the medications until it is time for them to take their dose, and at that time they will only be able to access the correct dosage which is able to be updated at a medical professional's, e.g., a doctor's, discretion. In particular embodiments, the systems of the present invention allow a medical professional (e.g., a doctor) to alter, in real-time, the patient's prescription based on the information the medical professional receives, e.g., from a vitals tests.

A. System Operation

When a medical professional, e.g., a doctor, prescribes medication, they enter into the system software a basic set of information on behalf of their patient. Generally, the medical professional provides information regarding the name of the medication, the correct number of pills to be taken at once, the strength of the medications, and finally, the time interval in between doses. The user, once they receive the medication from their pharmacy, which inserts the medications into the correct reservoir. Once all of the medications have been inserted, the machine locks and can no longer be opened. The process is simple, time-efficient, and user-friendly.

After the initial insertion of medications, a medical professional, and the medical professional only, has remote access to the software to program how often a medication needs to be dispensed and other necessary details (how many medications dispensed at a time, take with water, take with food, do not lift heavy objects for 30 minutes after taking, etc.). If necessary, the medical professional also has the ability to alter the prescription. If the dosage or time-intervals need to be increased or decreased, all the medical professional has to do is enter the changes into the software. The machine will reflect the changes within minutes.

In terms of the actual medication dispensing, the process of the dispensing operates efficiently and effectively. When it is time for the user to take a dose, the software will send an alert to the user's phone/watch that the medical professional and user pair with the machine before leaving the medical professional's office. In certain embodiments, the first alert tells the user that it is 30 minutes until a medication needs to be taken. That alert will stay up until 15 minutes later, when a 15-minute warning is given through the same alert system. That alert will stay up for 5 minutes, and there is another alert at 10 minutes until the medication is dispensed, and another one at 5. Then when the medication is dispensed, the user gets an alert along with the aforementioned necessary details. Immediately after the medication is taken, the machine's screen will open up to show attached machinery for the user to take vitals. In particular embodiments, these will be attached to the machine in order to ensure nothing is misplaced. In specific embodiments, the vitals being taken may be selected from one or more of the following: blood pressure (sphygmomanometer), body temperature (thermometer), heart rate (pulse oximeter), and respiration rate (capnography monitor).

In certain embodiments, the screen on the machine will display instructions as to how to operate the machinery. The medical professional may show the user how to operate the system, but further detailed instructions may be shown. If the machine recognizes that all vitals are taken, the information will be shared with the doctor/medical professional. In certain embodiments, if the vitals cross a certain threshold, the machine will deem the alert code red (which may be clear from the notification) and go straight to the medical professional. Otherwise, they will be normally sent for the medical professional to review. In particular embodiments, a weekly chart, e.g., a line graph, of the vitals will also be sent to the doctor.

In certain embodiments, if the machine does not recognize that vitals are taken, it will make an alarm noise with a Siri/Alexa like voice clearly indicating that vitals need to be taken. In certain embodiments, this will repeat once a minute for 10 minutes. If vitals are taken before then, all is normal. If vitals have not been taken, a louder, siren-like noise will play for 5 minutes. In particular embodiments, the alarms will be user-friendly in terms of how easy it is to shut it off.

### EXEMPLIFICATION

Having thus described the invention in general terms, reference will now be made to exemplary embodiments, and the accompanying drawings of exemplary embodiments, which are not necessarily drawn to scale, and which are not intended to be limiting in any way.

In this respect, it is to be understood that the invention is not limited in its application to the details of construction and to the arrangements of the components set forth in the following description or illustrated in the Figures. The invention is capable of other embodiments and of being practiced and carried out in various ways. Also, it is to be understood that the phraseology and terminology employed herein are for the purpose of description and should not be regarded as limiting.

### Example 1

### Exemplary Embodiments

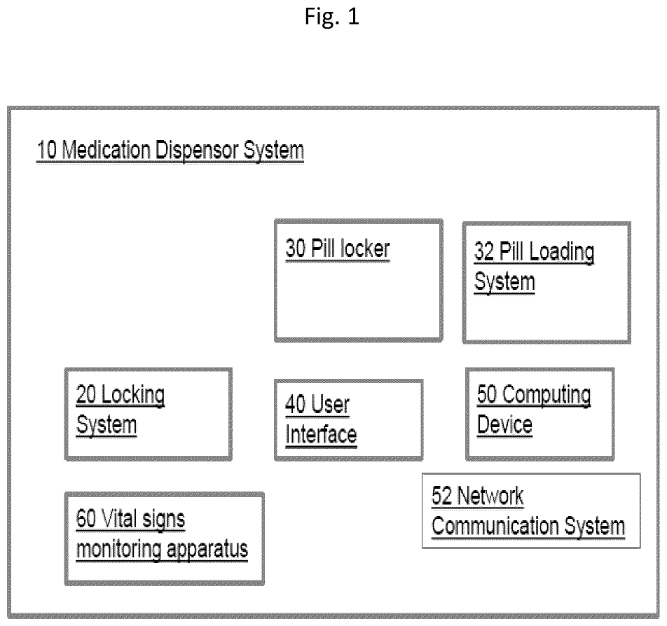

FIG. 1 illustrates an exemplary embodiment of the disclosed Medication Dispenser System 10, e.g., a Pill Dispensing System, includes a locking mechanism system 20. The lock mechanism 20 may be controlled by, for example a physical lock and key system, an electronic lock, a username/password combination, or similar access control device. The lock mechanism controls various aspects of the system 10. The lock mechanism controls access to the medicine by the patient, the lock mechanism also controls access to the medication loading system 32. The loading system is used as an access point to deposit medicine into the medication locker 30 of the system. Note that in some embodiments of the present disclosure there may be multiple medication lockers, e.g., pill lockers, within the system. In addition, the loading system allows a user of the system to deposit metadata associated with the medicine in electronic form into the device. This metadata may include, for example, information regarding the medicine formulation, the dosage associated with the medicine, contraindications associated with the medicine, and other information necessary to effectively run the system.

FIG. 1 further illustrates a user interface 40 that allows a user to enter and receive data to or from the system. The system 10 further includes a computing device 50 that is integrated into the system to store data and to provide processing decisions. The computing device is connected to a networked communication system 52 that transmits and receives messages from outside of the system. The networked communication system may, for example, use ethernet, 802.11, cellular communications or other means for external communications. The system further includes a vital sign monitoring apparatus 60. This system may access and record vitals such as Heart Rate, Blood Pressure, Blood Sugar, Body Temperature, or similar vital signs as necessary.

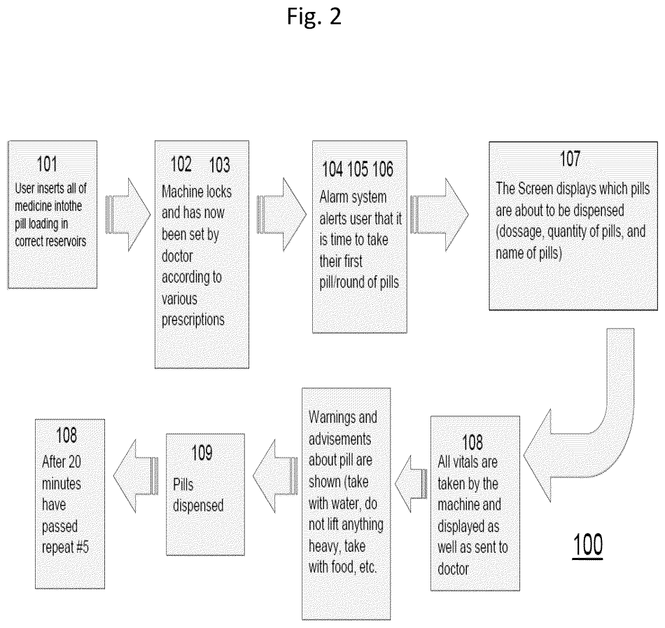

FIG. 2 illustrates a flow chart 100 in accord with the current disclosure where a user receives medicine from the medicine dispenser. In step 101 a user of the system adds medicine into the medication loading system 32 and provides metadata to the system 10 that corresponds to the medicine loaded in the machine. In step 102 the machine locks the medication locker. In Step 102/103 the machine executes a lock of the Medication Locker 30 and transmits a message to an interested party (e.g., a health care professional or pharmacy) to indicate that the medicine has been loaded in the machine.

In Step 104/105/106 the computing device 50 generates an alert to indicate that medicine should be dispensed. Such an alert may be generated based on: elapsed time since a last dosage was transmitted, an external signal received from a health care professional or a wearable health monitoring device, a schedule of dosages that has been input into the device either at the time that the medicine was supplied or at a later time via an email or similar transmission. The alert may consist of an audible message, a visible message, an email or text, or any similar means of informing a user of the system that an activity is required. It should be noted that when an alert is transmitted it may be sent to multiple recipients. Additional recipients may include, for example, a partner of the user, a healthcare professional, a parole officer, or any other interested party. Such parties will, typically, be required to have informed the user and to have provided the user's consent to the access to the information.

In Step 107 the user interface 40 provides information as to the medications to be dispensed. This information may include the quantity of medicine, dosage, medication type, as well as any contraindications that are related to the medicine being dispensed. In certain embodiments, Step 108 is performed wherein the user interacts with the vital signs monitoring apparatus to have certain vital signs recorded by the machine. The information collected in this step may be stored in the machine. It may also be transmitted to external resources for storage, analysis or other medically relevant activities. In certain embodiments the user interface may provide advertisements or informational content to support the individual in their health-related activities and/or their activities of daily living. In step 109 the medications are dispensed to the user.

In certain embodiments, a user is alerted to return to the device to have their vital signs monitored. The alert may be sent to the user at a time that is preset based on the dosage of medicine, type of medicine, a message received from a health care provider, vital sign information recorded in a previous session, or other relevant information.

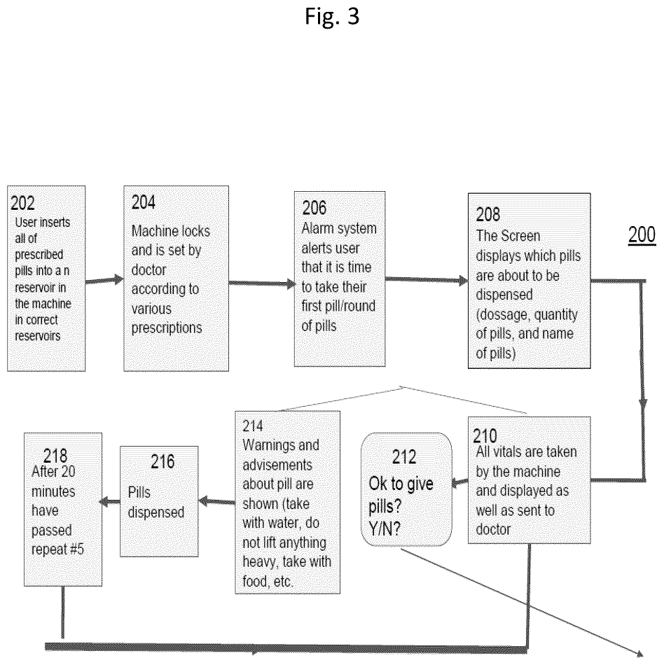

FIG. 3 illustrates a flowchart 200 where medicine is loaded into the dispenser. In Step 202 a user inserts medicine into a reservoir of the machine. Access to the reservoir may be controlled by a password, an electronic key, a mechanical key or other means of access control. In Step 204 the system locks access to the reservoir. At that time the system may also receive metadata relating to the medicine and/or the requirements that must be followed to safely use it. The system may also receive information that will be used in the recording of vital signs. Such information may relate to the frequency with which the vital signs are taken, the types of vital signs, the specific health care providers to be informed in the event of an episode that will be transmitted to a health care provider. In Step 206 the alarm system alerts the user that it is time to receive medicine from the device. In Step 208 the user interface displays information about the medicine to be dispensed. Such information may include, for example, dosage information and information relevant to the patient regarding the medicine. In step 210 the system measures the vital signs of the patient. In this step the system may simply record the data and/or transmit the information to a health care provider.

In Step 212 the system determines whether the medicine should be provided to the user. In Step 214 information is provided to the user. This information may include warning information relevant to the medicine, advertisements, informational content or the like. In Step 216 medicine is dispensed to the user. In step 218 the user may be called to the medicine dispenser for a follow up measurement of vital signs.

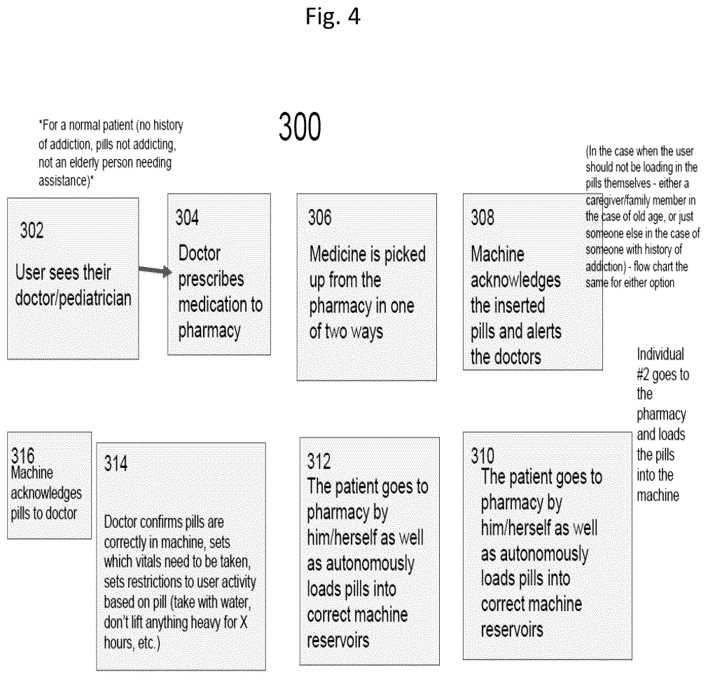

In FIG. 4, flow chart 300 is show which illustrates the disclosed system being used in the context of a healthcare provider's monitoring of a user's health. In step 302 a user initiates a process of obtaining medicine suitable for distribution through the disclosed medication dispensing system. In step 304 a healthcare provider specifies a medicine to be administered to the user. With the specification the healthcare provider may also provide dosage information, contraindications, and/or additional content to be provided to the user. In Step 306 medicine is provided to the user by a pharmacy or similar dispenser of medicine. In some embodiments of the disclosed system the medicine may be provided in a sealed container. Such a container may only be opened inside of the medicine dispensing system. This may be achieved through various means that will be familiar to a person skilled in the art. For example, there may be a magnetic lock that is releases only when the medication container is inside of the reservoir of the machine. In Step 308 the disclosed system receives the medicine and records the information related to the medicine. In certain embodiments the disclosed system may transmit a message that indicates that it has received the medicine. In Step 310 the patient may receive an additional medicine from pharmacy that may be stored in the medicine dispensing system in a second reservoir. In Step 312 the user may repeat the step for another medicine to be loaded in the dispenser.

In Step 314 a healthcare provider confirms that the medicine is correctly loaded in the machine. At this stage the healthcare provider may also load (or confirm the correct loading of) information related to the vital signs to be monitored or other similar requirements. In Step 316 the system confirms it is correctly functioning and continues its process.

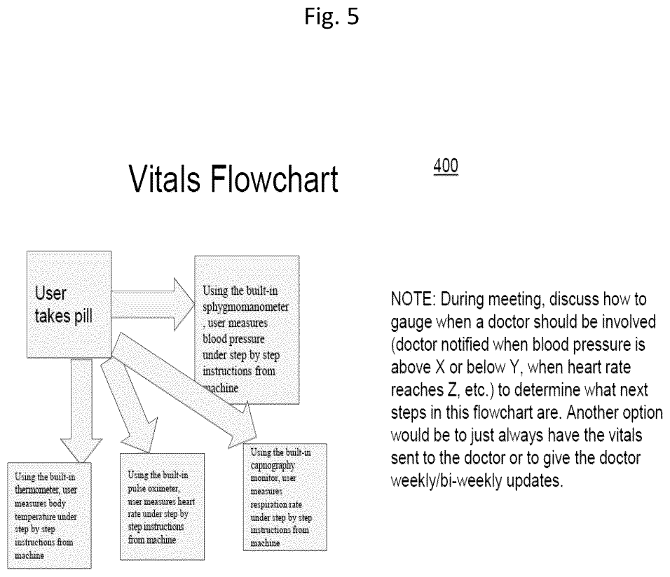

FIG. 5 provides an illustration 400 of matters relating to the collection of vital signs information by the disclosed system. The monitoring of vital signs process is, in certain embodiments, initiated by the user accessing medicine through the disclosed system. The vital signs monitoring system may, for example, include a sphygmomanometer for accessing and recording the patient's blood pressure. The monitoring of vital signs may also include a capnograph for monitoring the respiration rate of a user who is using the system. The monitoring of vital signs system may also include a pulse blood oximeter for determining the blood oxygen content of the user. The vital signs monitoring system may also include a thermometer for monitoring the user's temperature. Various other means of recording a user's state of health may be used as part of the vital signs monitoring system.

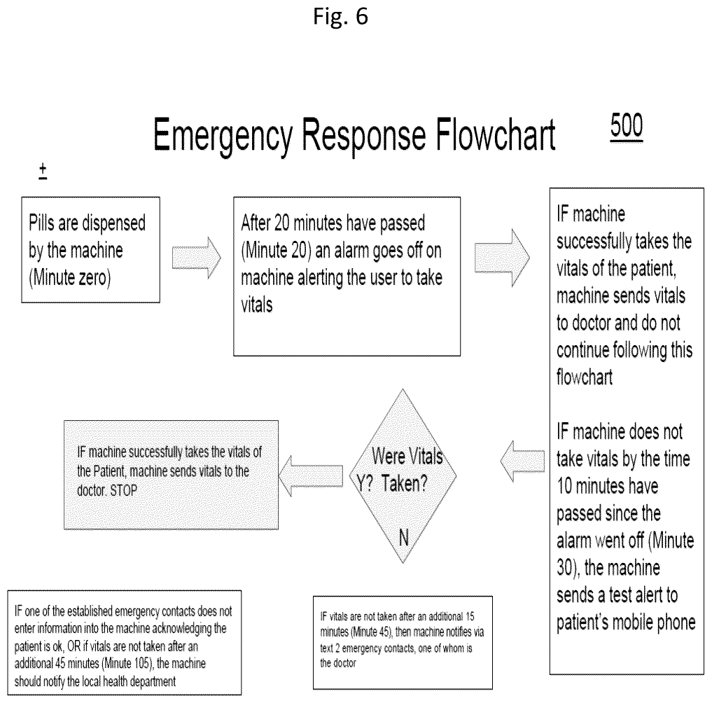

FIG. 6 provides an illustration of an emergency response flowchart 500. This flowchart illustrates a process that may be initiated in certain instances where an event (such as an adverse drug reaction) occurs for a user. In the first instance medicine is dispensed from the disclosed medicine dispensing system. After a period of time (in the flow chart show the period is twenty minutes) the medicine dispensing system triggers an alarm that may alert the user (and others that the user has been called to the medicine dispensing machine to have their vital signs monitored. In normal functioning of the system, the vital signs monitoring system will be utilized to monitor, record, and possibly transmit vital signs information for the user. In the event that the user does not return to the machine within the specified time to have their vital signs monitored there may be an issue. In such a case the medicine dispensing system will be triggered (after waiting for a fixed period that is specified in the records) the system may initiate an additional alert or it may trigger a response that includes informing emergency personnel, family or friends, and/or healthcare providers.

### INCORPORATION BY REFERENCE

The entire contents of all patents, published patent applications and other references cited herein are hereby expressly incorporated herein in their entireties by reference.

### EQUIVALENTS

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. An instrumented medication dispenser for monitoring usage of medication and adaptively enforcing compliance with a medication schedule comprising:
a user interface configured to receive a request for medication and to transmit that request to a processor;
a medication locker configured to provide access to a dosage of medicine, the dosage of medicine having associated prescription information; and
a computing device configured to receive the request for medication and to store information regarding the request, the computing device further configured to communicate a release request with the medication locker to release the dosage of medicine, wherein the computing device determines whether to transmit the release request based on a time of the request, the prescription information, and information regarding a previous request for the dosage.
